(19) United States
(12) Patent Application Publication
(10) Pub. No.: US 2024/0249818 A1
(43) Pub. Date: Jul. 25, 2024

(54) SYSTEM AND METHOD FOR GENERATING A PULMONARY DYSFUNCTION FUNCTIONAL PROGRAM

(71) Applicant: KPN INNOVATIONS LLC, LAKEWOOD, CO (US)

(72) Inventor: Kenneth Neumann, LAKEWOOD, CO (US)

(73) Assignee: KPN INNOVATIONS LLC, LAKEWOOD, CO (US)

(21) Appl. No.: 18/625,363

(22) Filed: Apr. 3, 2024

Related U.S. Application Data
(63) Continuation-in-part of application No. 17/387,325, filed on Jul. 28, 2021, now Pat. No. 11,955,224.
Application No. 17/387,325 is a continuation-in-part of application No. 17/136,087, filed on Dec. 29, 2020, now Pat. No. 11,145,400.

(51) Int. Cl. G16H 20/60; G16H 40/67

(52) U.S. Cl. CPC G16H 20/60; G16H 40/67

(57) ABSTRACT

A system for generating a pulmonary dysfunction nourishment program including computing device configured to receive at least a respiratory volume collection relating to a user, wherein the at least respiratory volume collection includes at least a biomarker, wherein the at least biomarker includes a bioremnant and a volatile organic compound (VOC), receive a salubrious reference relating to the user, identify a functional signature as a function of the salubrious reference, wherein the identifying further includes receiving a conduct indicator wherein the conduct indicator includes an indicator index, generating one or more conduct parameters as function of the salubrious reference, comparing the conduct indicator to the one or more conduct parameters and identifying the functional signature as a function of the comparison, and generate a nourishment program as a function of the functional signature.

### CROSS-REFERENCE TO RELATED APPLICATIONS

This application is a continuation-in-part of Non-provisional application Ser. No. 17/387,325 filed on Jul. 28, 2021, and entitled “SYSTEM AND METHOD FOR GENERATING A PULMONARY DYSFUNCTION FUNCTIONAL PROGRAM,” which is a continuation-in-part of Non-provisional application Ser. No. 17/136,087 filed on Dec. 29, 2020, now U.S. Pat. No. 11,145,400 issued on Oct. 12, 2021, and entitled “SYSTEM AND METHOD FOR GENERATING A PULMONARY DYSFUNCTION NOURISHMENT PROGRAM,” the entirety of which are incorporated herein by reference.

### FIELD OF THE INVENTION

The present invention generally relates to the field of artificial intelligence. In particular, the present invention is directed to a system and method for generating a pulmonary dysfunction nourishment program.

### BACKGROUND

Current edible suggestion systems do not account for pulmonary characteristics of an individual. This leads to inefficiency of an edible suggestion system and a poor nutrition plan for the individual. This is further complicated by a lack of uniformity of nutritional plans, which results in dissatisfaction of individuals.

### SUMMARY OF THE DISCLOSURE

In an aspect a system for generating a pulmonary dysfunction nourishment program is described. The system includes a computing device. The computing device is configured to receive at least a respiratory volume collection relating to a user, wherein the at least respiratory volume collection includes at least a biomarker, wherein the at least biomarker includes a bioremnant and a volatile organic compound (VOC), receive a salubrious reference relating to the user, identify a functional signature as a function of the salubrious reference, wherein the identifying further includes receiving a conduct wherein the conduct indicator includes an indicator index, comparing the conduct indicator to one or more conduct parameters, generating the one or more conduct parameters as function of the salubrious reference, and identifying the functional signature as a function of the comparison, and a functional machine-learning model and generate a program as a function of the functional signature.

In another aspect a method for generating a pulmonary dysfunction functional program is described. The method includes receiving, by a computing device, at least a respiratory volume collection relating to a user, wherein the at least respiratory volume collection includes at least a biomarker, wherein the at least biomarker includes a bioremnant and a volatile organic compound (VOC), receiving, by the computing device, a salubrious reference relating to the user, identifying, by the computing device, a functional signature as a function of the salubrious reference, wherein the identifying further includes receiving a conduct indicator wherein the conduct indicator includes an indicator index, generating one or more conduct parameters as function of the salubrious reference, comparing the conduct indicator to the one or more conduct parameters, and identifying the functional signature as a function of the comparison and generating, by the computing device, a nourishment program as a function of the functional signature.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for generating a pulmonary dysfunction nourishment program;

FIG. 2 is a block diagram illustrating an exemplary embodiment of a system for generating a pulmonary dysfunction functional program;

FIG. 3 is a representative diagram of an exemplary embodiment of respiratory parameters according to an embodiment of the invention;

FIG. 4 is a block diagram of an exemplary embodiment of an edible directory according to an embodiment of the invention;

FIG. 5 is a representative diagram of an exemplary embodiment of biomarkers that can be received from a respiratory volume collection according to an embodiment of the invention;

FIG. 6 is a block diagram of an exemplary embodiment of a machine-learning module;

FIG. 7 is a process flow diagram illustrating an exemplary embodiment of a method of generating a pulmonary dysfunction nourishment program;

FIG. 8 is a process flow diagram illustrating an exemplary embodiment of a method of generating a pulmonary dysfunction functional program;

FIG. 9 is yet another exemplary embodiment of a method of generating a pulmonary dysfunction functional program; and

FIG. 10 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating a pulmonary dysfunction functional program. In an embodiment, the disclosure may receive at least a respiratory volume relating to a user. Aspects of the present disclosure can be used to produce at least a respiratory parameter as a function of the respiratory volume collection using respiratory algorithms. Aspects of the present disclosure can be used to identify a functional signature as a function of the pulmonary bundle element. This is so, at least in part, because disclosure utilizes a functional machine-learning model. Aspects of the present disclosure allow for generating a functional program. Exemplary embodiments illustrating aspects of the present disclosure are described below in the context of several specific examples.

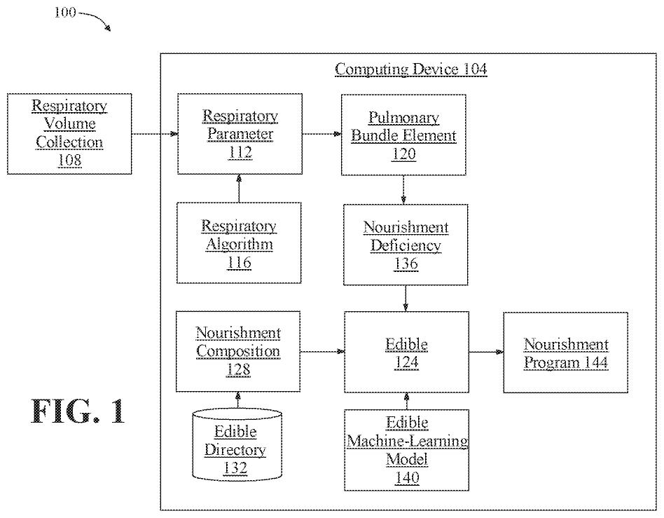

Referring now to FIG. 1, an exemplary embodiment of a system 100 for generating a pulmonary dysfunction nourishment program is illustrated. System includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

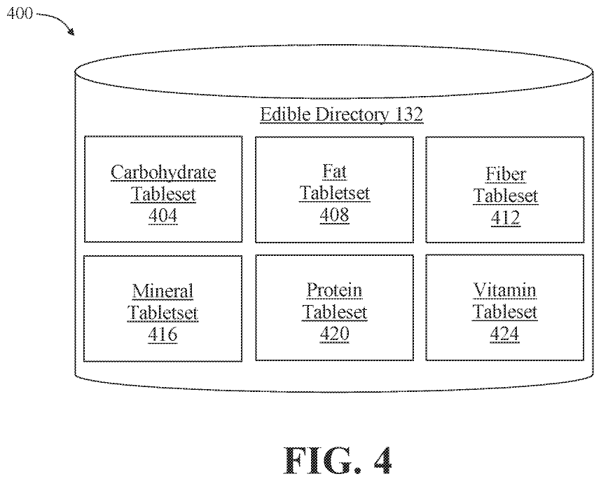

Still referring to FIG. 1, computing device 104 receives at least a respiratory volume collection 108 relating to a user. As used in this disclosure “respiratory volume collection” is a volume of biological sample from an individual associated with the respiratory system of an individual. A biological sample may include, without limitation, exhalate, blood, sputum, urine, saliva, feces, semen and other bodily fluids, as well as tissue. Exhalate may include, without limitation, breath that is expelled from an individual via breathing and/or some other forced exhalation of breath from an individual. As a non-limiting example respiratory volume collection 108 may include a volume of 2.5 L of breath exhaled from an individual. As a further non-limiting example, respiratory volume collection 108 may include a volume of 0.1 L of blood from the pulmonary system of an individual. Respiratory volume collection 108 may include expired breath from any pulmonary entry and/or exit pathway, including but not limited to oral and/or nasal passages. Respiratory volume collection 108 may be received as a function of obtaining a respiratory signal from at least a sensor. As used in this disclosure “respiratory signal” is datum that relates to and/or represents an element associated with the status of an individual's respiratory system. As a non-limiting example a respiratory signal may include an image of a lung of an individual from a magnetic resonance imaging medical device. As a further non-limiting example a respiratory signal may include one or more lights, voltages, currents, sounds, chemicals, pressures, moistures, and the like thereof. Respiratory signal may include one or more respiratory biomarkers associated with the pulmonary system of an individual, wherein a biomarker is one or more chemicals, components, molecules, gases, and the like there of as described in detail below, in reference to FIG. 4. As used in this disclosure “sensor” is a device that records, monitors, stores, measures, and/or transmits respiratory signals. As a non-limiting example, a sensor may include an imaging sensor, such as optical cameras, infrared cameras, 3D cameras, multispectral cameras, hyperspectral cameras, polarized cameras, chemical sensors, motion sensors, ranging sensors, light radar component, such as lidar, detection or imaging using radio frequencies component, such as radar, terahertz or millimeter wave imagers, seismic sensors, magnetic sensors, weight/mass sensors, ionizing radiation sensors, and/or acoustical sensors. As a further non-limiting example, a sensor may include one or more medical devices that at least detect and/or monitor an individual's respiratory system, such as semi-auto analyzers, photo colorimeters, cell photo colorimeters, hemoglobin meters, mass spectrometers, chromatographic instruments, and the like thereof. In one or more embodiments, respiratory volume collection 108 may be received from one or more medical entities. In one or more embodiments, medical entities may include medical professionals and companies associated with the medical field. In one or more embodiments, medial entities may perform examinations, laboratory testing and the like wherein computing device may be configured to receive the results from medical entities. In one or more embodiments, respiratory volume collection 108 may be iteratively received from one or more medical entities wherein computing device 104 may be configured to iteratively receive respiratory volume collection 108 and update functional signature and nourishment program. In one or more embodiments, a patient may undergo periodic lab testing wherein respiratory volume collection 108 may be received from the lab testing. In one or more embodiments, respiratory volume collection 108 may be iteratively received wherein following treatment by a medical professional, a medical professional may receive respiratory volume collection 108 and update system 100. In one or more embodiments, respiratory volume may be received in any way as described in this disclosure may be iteratively received from one or more input devices configured to receive respiratory volume. In one or more embodiments, computing device 104 may notify user to periodically input respiratory volume collection 108 either through text input and/or through one or more sensing devices configured to receive respiratory volume collection 108. In one or more embodiments, computing device 104 may utilize an API to receive lab results from medical entities. In one or more embodiments, medical entities may be periodically notified for receipt of information wherein medical entities may transmit respiratory volume collection 108 to computing device 104. In one or more embodiments, respiratory volume collection 108 may include laboratory results which may be used to iteratively and/or continuously track a user's health.

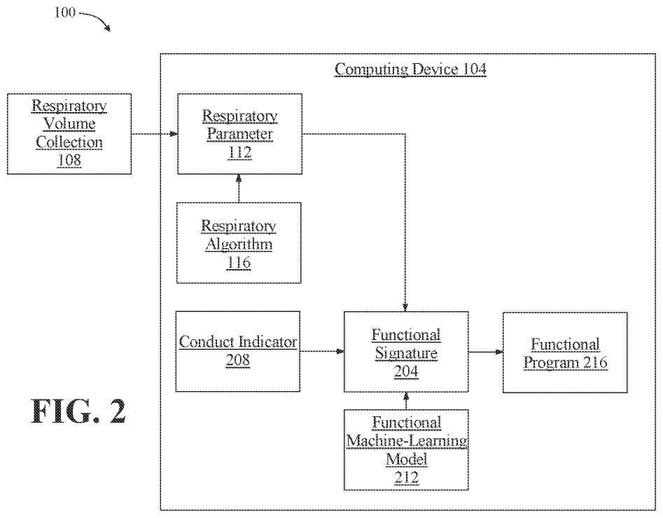

Still referring to FIG. 1, computing device 104 generates at least a respiratory parameter 112 of a plurality of respiratory parameters as a function of respiratory volume collection 108. As used in this disclosure “respiratory parameter” is a measurable value associated with an individual's respiratory system. As a non-limiting example respiratory parameter may include one or more chemical concentrations, rates of flow, lung volumes, diffusion capacities, and the like there of. As a further non-limiting example a respiratory parameter may include, without limitation, an inspiratory reserve volume (IRV), tidal volume (TV), expiratory reserve volume (ERV), residual volume (RV), inspiratory capacity (IC), functional residual capacity (FRC), vital capacity (VC), total lung capacity (TLC), as described in detail below, in reference to FIG. 2. Respiratory parameter 112 is generated as a function of a respiratory algorithm 116. As used in this disclosure “respiratory algorithm” is an algorithm that determines one or more respiratory measurements. As a non-limiting example, respiratory algorithm 116 may include algorithms such as a minute ventilation, alveolar minute ventilation, airway resistance, mean airway pressure, work of breathing, alveolar-arterial oxygen tension gradient, alveolar oxygen tension, arterial/alveolar oxygen tension, arterial oxygen content, end-capillary oxygen content, mixed venous oxygen content, shunt equation, modified shunt equation, arterial-mixed venous oxygen content difference, oxygen-to-air entrainment ratio, arterial oxygen saturation estimation, P/F ratio, oxygenation index, oxygen consumption, oxygen extraction ratio, fiO2 estimation for nasal cannula, oxygen cylinder duration, liquid oxygen system duration, cardiac index, cardiac output, cardiac output Fick's method, cerebral perfusion pressure, mean arterial pressure, stroke volume, maximum heart rate, heart rate on an EKG strip, respiratory quotient, systemic vascular resistance, pulmonary vascular resistance, static compliance, dynamic compliance, dead space to tidal volume ratio, children dosage estimation, infant dosage estimation, infant and children dosage estimation, anion gap, body surface area elastance, smoking use calculation, suction catheter size estimation, endotracheal tube size estimation in children, Boyle's law, Charles' law, Gay-Lussac's law, LaPlace's law, Celsius to Fahrenheit temperature conversion, Fahrenheit to Celsius temperature conversion, Celsius to Kelvin temperature conversion, helium/oxygen conversion, total lung capacity, pressure support ventilator setting, rapid shallow breathing index, endotracheal tube size estimation in children, minimum flow rate in mechanical ventilation, and the like thereof.

Still referring to FIG. 1, computing device 104 determines a pulmonary bundle element 120 as a function of respiratory parameter 116. As used in this disclosure “pulmonary bundle element” is a profile of a user's respiratory status consisting of a group of respiratory parameters. As a non-limiting example pulmonary bundle element 120 may group respiratory parameters of oxygen saturation, cardiac index, tidal volume, and oxygen cylinder duration. Computing device 104 may determine pulmonary bundle element 120 by identifying at least a pulmonary deficiency as a function of the respiratory parameter. As used in this disclosure “pulmonary deficiency” is an inadequacy and/or deficiency of a respiratory parameter. As a non-limiting example a pulmonary deficiency may exist due to a respiratory rate of 20, wherein a respiratory rate should be 40 according to a respiratory threshold. As used in this disclosure “respiratory threshold” is a threshold a respiratory parameter should be. Respiratory threshold may be identified according to one or more medical guidelines for the measurement of respiratory function. As a non-limiting example a medical guideline for the measurement of respiratory function may include a defined threshold according to the American Association for Respiratory Care, American Medical Association, American College of Physicians, and the like thereof. As a further non-limiting example, a medical guideline for the measurement of respiratory function may include a defined threshold according to one or more medical research journals, such as the Lancet, New England Journal of Medicine, Science, Journal of the American Medical Association, and the like thereof.

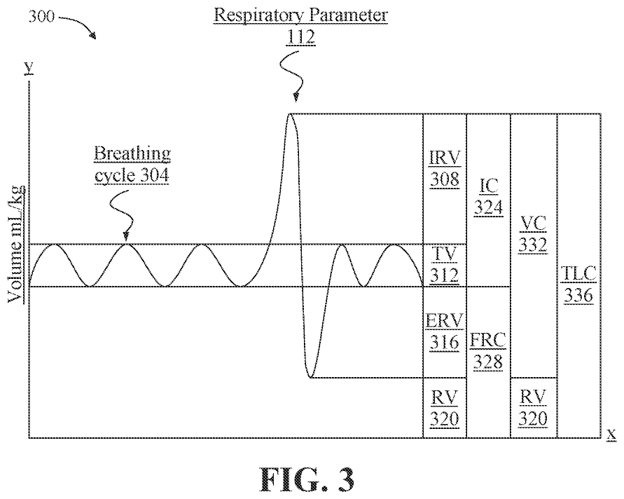

Still referring to FIG. 1, computing device 104 identifies at least an edible as a function of pulmonary bundle element 120. As used in this disclosure an “edible” is a source of nourishment that may be consumed by a user such that the user may absorb the nutrients from the source. For example and without limitation, an edible may include legumes, plants, fungi, nuts, seeds, breads, dairy, eggs, meat, cereals, rice, seafood, desserts, dried foods, dumplings, pies, noodles, salads, stews, soups, sauces, sandwiches, and the like thereof. Computing device 104 identifies edible 124 as a function of obtaining a nourishment composition 128. As used in this disclosure “nourishment composition” is a list and/or compilation of all of the nutrients contained in an edible. As a non-limiting example nourishment composition 128 may include one or more quantities and/or amounts of total fat, including saturated fat and/or trans-fat, cholesterol, sodium, total carbohydrates, including dietary fiber and/or total sugars, protein, vitamin A, vitamin C, thiamin, riboflavin, niacin, pantothenic acid, vitamin b6, folate, biotin, vitamin B12, vitamin D, vitamin E, vitamin K, calcium, iron, phosphorous, iodine, magnesium, zinc, selenium, copper, manganese, chromium, molybdenum, chloride, and the like thereof. Nourishment composition 128 may be obtained as a function of an edible directory 132, wherein an edible directory is a database of edibles that may be identified as a function of one or more pulmonary bundle elements, as described in detail below, in reference to FIG. 3. Computing device 104 determines a nourishment deficiency 136 as a function of pulmonary bundle element 120. As used in this disclosure “nourishment deficiency” is an inadequacy and/or deficiency of a nutrient in a user's body. As a non-limiting example pulmonary bundle element 120 may determine a reduced hemoglobin concentration, wherein a nourishment deficiency may be identified as low iron. Nourishment deficiency 136 may be identified according to one or more nourishment guidelines. As a non-limiting example a nourishment guideline may be identified according to a peer-review research journal, such as the Journal of Nutrition, Nutrition and Health, Advances in Nutrition, and the like thereof.

Still referring to FIG. 1, computing device 104 identifies edible 124 as a function of nourishment composition 128, nourishment deficiency 136, and an edible machine-learning model 140. As used in this disclosure “edible machine-learning model” is a machine-learning model to produce an edible output given nourishment compositions and nourishment deficiencies as inputs; this is in contrast to a non-machine learning software program where the commands to be executed are determined in advance by a user and written in a programming language. Edible machine-learning model 140 may include one or more edible machine-learning processes such as supervised, unsupervised, or reinforcement machine-learning processes that computing device 104 and/or a remote device may or may not use in the determination of edible 124. As used in this disclosure “remote device” is an external device to computing device 104. An edible machine-learning process may include, without limitation machine learning processes such as simple linear regression, multiple linear regression, polynomial regression, support vector regression, ridge regression, lasso regression, elastienet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train edible machine-learning process as a function of an edible training set. As used in this disclosure a “edible training set” is a training set that correlates at least nourishment composition and nourishment deficiency to an edible. For example, and without limitation, nourishment composition of 14 g of protein and 2 g of fiber and a nourishment deficiency of low levels of protein CC16 as a function of chronic obstructive pulmonary disease may relate to an edible of salmon. The edible training set may be received as a function of user-entered valuations of nourishment compositions, nourishment deficiencies, and/or edibles. Computing device 104 may receive edible training set by receiving correlations of nourishment compositions and/or nourishment deficiencies that were previously received and/or determined during a previous iteration of determining edibles. The edible training set may be received by one or more remote devices that at least correlate a nourishment composition and nourishment deficiency to an edible, wherein a remote device is an external device to computing device 104, as described above.

Still referring to FIG. 1, edible machine-learning model 140 may identify edible 120 as a function of one or more classifiers. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 104 and/or another device may generate a classifier using a classification algorithm, defined as a processes whereby a computing device 104 derives a classifier from training data. Classification may be performed using, without limitation, linear classifiers such as without limitation logistic regression and/or naïve Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least one value. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {{a}_{i}}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, computing device 104 may receive edible machine-learning model 140 from the remote device that utilizes one or more edible machine learning processes, wherein a remote device is described above in detail. For example, and without limitation, a remote device may include a computing device, external device, processor, and the like thereof. The remote device may perform the edible machine-learning process using the edible training set to generate edible 124 and transmit the output to computing device 104. The remote device may transmit a signal, bit, datum, or parameter to computing device 104 that at least relates to edible 124. Additionally or alternatively, the remote device may provide an updated machine-learning model. For example, and without limitation, an updated machine-learning model may be comprised of a firmware update, a software update, an edible machine-learning process correction, and the like thereof. As a non-limiting example a software update may incorporate a new nourishment composition that relates to a modified nourishment deficiency. Additionally or alternatively, the updated machine learning model may be transmitted to the remote device, wherein the remote device may replace the edible machine-learning model with the updated machine-learning model and determine the edible as a function of the nourishment deficiency using the updated machine-learning model. The updated machine-learning model may be transmitted by the remote device and received by computing device 104 as a software update, firmware update, or corrected edible machine-learning model. For example, and without limitation an edible machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference.

Still referring to FIG. 1, computing device 104 may identify edible as a function of determining a pulmonary dysfunction. As used in this disclosure “pulmonary dysfunction” is an ailment and/or collection of ailments that impact an individual's respiratory system. As a non-limiting example, pulmonary dysfunctions may include asthma, chronic obstructive pulmonary disorder, chronic bronchitis, emphysema, lung cancer, cystic fibrosis, pneumonia, pleural effusion, acute bronchitis, pulmonary edema, sarcoidosis, asbestosis, autoimmune pulmonary alveolar proteinosis, Blau syndrome, bronchogenic cysts, Cantu syndrome, Gaucher disease, Henoch-Schonlein purpura, idiopathic pulmonary fibrosis, and the like thereof. Pulmonary dysfunction may be determined as a function of one or more pulmonary machine-learning models. As used in this disclosure “pulmonary machine-learning model” is a machine-learning model to produce a pulmonary dysfunction output given pulmonary bundle elements as inputs; this is in contrast to a non-machine learning software program where the commands to be executed are determined in advance by a user and written in a programming language. Pulmonary machine-learning model may include one or more pulmonary machine-learning processes such as supervised, unsupervised, or reinforcement machine-learning processes that computing device 104 and/or a remote device may or may not use in the determination of pulmonary dysfunction. As used in this disclosure “remote device” is an external device to computing device 104. A pulmonary machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train pulmonary machine-learning process as a function of a pulmonary training set. As used in this disclosure a “pulmonary training set” is a training set that correlates at least ventilatory enumeration and ventilatory effect to a pulmonary dysfunction. As used in this disclosure “ventilatory enumeration” is a measurable value associated with a ventilatory system and/or respiratory system. As used in this disclosure “ventilatory effect” is an impact and/or effect on the pulmonary system of an individual. As a non-limiting example a ventilatory enumeration of 20 may be established for a ventilatory effect of shortness of breath, wherein a pulmonary dysfunction of COVID-19 may be determined. The pulmonary training set may be received as a function of user-entered valuations of ventilatory enumerations, ventilatory effects, and/or pulmonary dysfunctions. Computing device 104 may receive pulmonary training by receiving correlations of ventilatory enumerations and/or ventilatory effects that were previously received and/or determined during a previous iteration of determining pulmonary dysfunction. The pulmonary training set may be received by one or more remote devices that correlate a ventilatory enumeration and/or ventilatory effect to a pulmonary dysfunction, wherein a remote device is an external device to computing device 104, as described above.

Still referring to FIG. 1, computing device 104 may receive pulmonary machine-learning model from the remote device that utilizes one or more pulmonary machine learning processes, wherein a remote device is described above in detail. For example, and without limitation, a remote device may include a computing device, external device, processor, and the like thereof. The remote device may perform the pulmonary machine-learning process using the pulmonary training set to generate pulmonary dysfunction and transmit the output to computing device 104. The remote device may transmit a signal, bit, datum, or parameter to computing device 104 that at least relates to pulmonary dysfunction. Additionally or alternatively, the remote device may provide an updated machine-learning model. For example, and without limitation, an updated machine-learning model may be comprised of a firmware update, a software update, a pulmonary machine-learning process correction, and the like thereof. As a non-limiting example a software update may incorporate a new ventilatory enumeration that relates to a modified ventilatory effect. Additionally or alternatively, the updated machine learning model may be transmitted to the remote device, wherein the remote device may replace the pulmonary machine-learning model with the updated machine-learning model and determine the pulmonary dysfunction as a function of the ventilatory enumeration using the updated machine-learning model. The updated machine-learning model may be transmitted by the remote device and received by computing device 104 as a software update, firmware update, or corrected pulmonary machine-learning model. For example, and without limitation pulmonary machine-learning model may utilize a logistic regression machine-learning process, wherein the updated machine-learning model may incorporate decision tree machine-learning process.

Still referring to FIG. 1, computing device 104 may identify edible as a function of a likelihood parameter. As used in this disclosure “likelihood parameter” is a parameter that identities the probability of a user to consume an edible. As a non-limiting example likelihood parameter may identify a high probability that a user will consume an edible of steak. As a further non-limiting example likelihood parameter may identify a low probability that a user will consume an edible of cookies. Likelihood parameter may be determined as a function of a user taste profile. As used in this disclosure “user taste profile” is a profile of a user that identifies one or more desires, preferences, wishes, and/or wants that a user has. As a non-limiting example a user taste profile may include a user's preference for chicken flavor and/or crunchy textured edibles. Likelihood parameter may be determined as a function of an edible profile. As used in this disclosure “edible profile” is taste of an edible is the sensation of flavor perceived in the mouth and throat on contact with the edible. Edible profile may include one or more flavor variables. As used in this disclosure “flavor variable” is a variable associated with the distinctive taste of an edible, wherein a distinctive may include, without limitation sweet, bitter, sour, salty, umami, cool, and/or hot. Edible profile may be determined as a function of receiving flavor variable from a flavor directory. As used in this disclosure “flavor directory” is a database of flavors for an edible. As a non-limiting example flavor directory may include a list and/or collection of edibles that all contain umami flavor variables. As a further non-limiting example flavor directory may include a list and/or collection of edibles that all contain sour flavor variables. Likelihood parameter may alternatively or additionally include any user taste profile and/or edible profile used as a likelihood parameter as described in U.S. Nonprovisional application Ser. No. 17/032,080, filed on Sep. 25, 2020, and entitled “METHODS, SYSTEMS, AND DEVICES FOR GENERATING A REFRESHMENT INSTRUCTION SET BASED ON INDIVIDUAL PREFERENCES,” the entirety of which is incorporated herein by reference.

Still referring to FIG. 1, computing device 104 outputs a nourishment program 144 of a plurality of nourishment programs as a function of the edible 124. As used in this disclosure “nourishment program” is a program consisting of one or more edibles that are to be consumed over a given time period, wherein a time period is a temporal measurement such as seconds, minutes, hours, days, weeks, months, years, and the like thereof. As a non-limiting example nourishment program 144 may consist of recommending steak for 3 days. As a further non-limiting example nourishment program 144 may recommend chicken for a first day, spaghetti for a second day, and mushrooms for a third day. Nourishment program 144 may include one or more diet programs such as paleo, keto, vegan, vegetarian, and the like thereof. Nourishment program 144 may be outputted as a function an intended outcome. As used in this disclosure “intended outcome” is an outcome that an edible may generate according to a predicted and/or purposeful plan. As a non-limiting example, intended outcome may include a treatment outcome. As used in this disclosure “treatment outcome” is an intended outcome that is designed to at least reverse and/or eliminate the effects of the pulmonary bundle element and/or pulmonary dysfunction. As a non-limiting example, a treatment outcome may include reversing the effects of emphysema. As a further non-limiting example, a treatment outcome includes reversing the pulmonary dysfunction of lung fibrosis. Intended outcome may include a prevention outcome. As used in this disclosure “prevention outcome” is an intended outcome that is designed to at least prevent and/or avert a pulmonary bundle element and/or pulmonary dysfunction. As a non-limiting example, a prevention outcome may include preventing the development of chronic obstructive pulmonary disease.

Still referring to FIG. 1, computing device 104 may output nourishment program 144 as a function of the intended outcome using a nourishment machine-learning model. As used in this disclosure “nourishment machine-learning model” is a machine-learning model to produce a nourishment program output given edibles and/or intended outcomes as inputs; this is in contrast to a non-machine learning software program where the commands to be executed are determined in advance by a user and written in a programming language. Nourishment machine-learning model may include one or more nourishment machine-learning processes such as supervised, unsupervised, or reinforcement machine-learning processes that computing device 104 and/or a remote device may or may not use in the output of nourishment program 144. As used in this disclosure “remote device” is an external device to computing device 104. Nourishment machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train nourishment machine-learning process as a function of a nourishment training set. As used in this disclosure a “nourishment training set” is a training set that correlates an intended outcome to an edible. The nourishment training set may be received as a function of user-entered edibles, intendent outcomes, and/or nourishment programs. Computing device 104 may receive nourishment training by receiving correlations of intended outcomes and/or edibles that were previously received and/or determined during a previous iteration of outputted nourishment programs. The nourishment training set may be received by one or more remote devices that correlate an intended outcome and/or edible to a nourishment program, wherein a remote device is an external device to computing device 104, as described above.

Still referring to FIG. 1, computing device 104 may receive nourishment machine-learning model from the remote device that utilizes one or more nourishment machine learning processes, wherein a remote device is described above in detail. For example, and without limitation, a remote device may include a computing device, external device, processor, and the like thereof. The remote device may perform the nourishment machine-learning process using the nourishment training set to output nourishment program 144 and transmit the output to computing device 104. The remote device may transmit a signal, bit, datum, or parameter to computing device 104 that at least relates to nourishment program 144. Additionally or alternatively, the remote device may provide an updated machine-learning model. For example, and without limitation, an updated machine-learning model may be comprised of a firmware update, a software update, a nourishment machine-learning process correction, and the like thereof. As a non-limiting example a software update may incorporate a new intended outcome that relates to a modified edible. Additionally or alternatively, the updated machine learning model may be transmitted to the remote device, wherein the remote device may replace the nourishment machine-learning model with the updated machine-learning model and output the nourishment program as a function of the intended outcome using the updated machine-learning model. The updated machine-learning model may be transmitted by the remote device and received by computing device 104 as a software update, firmware update, or corrected nourishment machine-learning model. For example, and without limitation nourishment machine-learning model may utilize a nearest neighbor machine-learning process, wherein the updated machine-learning model may incorporate association rules machine-learning processes.

Now referring to FIG. 2, an exemplary embodiment of a system 200 for generating pulmonary dysfunction functional program is illustrated. System 200 includes computing device 104. Computing device 104 may include any computing device 104 as described above in detail, in reference to FIG. 1. For example and without limitation, computing device 104 may include a microcontroller, microprocessor, digital signal processor (DSP) and/or system on a chip (SoC) as described in this disclosure. Computing device 104 is configured to obtain a respiratory volume collection 108 relating to a user. Respiratory volume collection 108 includes any of the respiratory volume collection 108 as described above in detail, in reference to FIG. 1. For example, and without respiratory volume collection 108 may include a marker that represents a health status of a user's respiratory system such as, but not limited to a biological sample, biomarkers, respiratory signal, and the like thereof.

Still referring to FIG. 2, computing device 104 is configured to produce at least a respiratory parameter 112 of a plurality of respiratory parameters as a function of the at least a respiratory volume collection 108. Respiratory parameter 112 includes any of the respiratory parameter 112 as described above, in reference to FIG. 1. Computing device 104 generates respiratory parameter 112 using a respiratory algorithm 116. Respiratory algorithm 116 includes any of the respiratory algorithm 116 as described above, in reference to FIG. 1.

Still referring to FIG. 2, computing device 104 is configured to identify a functional signature 204 as a function of respiratory parameter 112. As used in this disclosure a “functional signature” is a profile representing an individual's relative measure of wellness. For example and without limitation, functional signature 204 may represent that an individual is “healthy” and/or in an excellent wellness state. As a further non-limiting example, functional signature 204 may represent that an individual has is “unhealthy” and/or in a poor wellness state. In an embodiment, and without limitation, computing device 104 may identify functional signature 204 as a function of determining a pulmonary dysfunction, wherein determining a pulmonary dysfunction is described above, in reference to FIG. 1. Pulmonary dysfunction includes any of the pulmonary dysfunction as described above, in reference to FIG. 1. Functional signature 204 is identified as a function of receiving a conduct indicator 208. As used in this disclosure a “conduct indicator” is an element of data denoting an individual's lifestyle choices. In an embodiment conduct indicator 208 may include one or more biological, psychological, social, and/or spiritual elements. For example, and without limitation, conduct indicator 208 may denote a biological element, wherein the biological indicator may denote that an individual has low cholesterol and/or exercises frequently. As a further non-limiting example, conduct indicator 208 may denote a psychological element, wherein the psychological element may denote that an individual is happy and/or content. In one or more embodiments, conduct indicator may be received from a remote device such as a wearable device. In one or more embodiments, wearable device may be configured to receive conduct indicator. In one or more embodiments, wearable device may receive electrocardiogram signals, blood oxygen levels, heart rate and the like. In one or more embodiments, data received from wearable device may be converted into conduct indicators wherein a particular heart rate, blood oxygen level and the like may be correlated to particular sleep habits, health habits and the like. In one or more embodiments, wearable devices may include but are not limited to, smartwatches, smart clothing, health monitoring patches, smart rings. Blood pressure monitors and the like. As a further non-limiting example, conduct indicator 208 may denote a social element, wherein the social element may indicate that an individual has 36 friends. As a further non-limiting example, conduct indicator 208 may denote a spiritual element, wherein the spiritual element may indicate that an individual belongs to the Hinduism religion. As a further non-limiting example, spiritual element may denote one or more chakras and/or spiritual energies of an individual. In an embodiment conduct indicator 208 may denote one or more lifestyles groups such as, but not limited to, general lifestyles, income, profession, and/or occupation lifestyles, consumption-based lifestyles, social and/or political lifestyles, marketing lifestyles, military lifestyles, sexual lifestyles, spiritual lifestyles, religious lifestyles, musical lifestyles, recreational lifestyles, and the like thereof. For example, and without limitation, lifestyles may include activism, asceticism, modern primitivism, bohemianism, communal living, clothes free, groupie lifestyle, hippie, quirkyalone, rural lifestyle, simple living, traditional lifestyle, criminality, farming, jet set, piracy, poverty, prostitution, sarariman, workaholic, yuppie, social liberalism, social conservatism, polygamy, monogamy, ahimsa, Hinduism, Christianity, evangelicalism, Islam, Judaism, missionary, Zen, yoga, Thelema, surfer, athleticism, hunter, artist, golf, recreational drug use, and the like thereof. Additionally or alternatively conduct indicator 208 may include one or more markers associated with an individual's behavior such as, but not limited to, markers identified respiratory volume collection 108. For example, and without limitation markers may include but are not limited to biological samples, biomarkers, respiratory signals, and the like thereof as defined above, in reference to FIG. 1.

With continued reference to FIG. 2, computing device may generate a graphical representation of functional signature and display graphical representation through a user interface. In one or more embodiments, graphical representation may include a visual presentation of data such as but not limited to in the form of an XY chart, a Venn diagram, a heatmap and the like. In one or more embodiments, graphical representation may include an XY chart wherein changes in functional signatures may be graphed over a given period of time in order to determine and/or visualize changes in a user's health. In one or more embodiments, computing device 104 may be configured to generate a graphical representation of the functional signature and a plurality of functional signatures received from previous iterations of the processing. In one or more embodiments, plurality of functional signatures may include functional signatures generated for a user on previous iterations of processing of system 100, such as for example, processing on a previous hour, month day, year and the like. In one or more embodiments, following each processing of system 100, functional signatures may be stored in database, wherein functional signatures stored in database may be referred to as plurality of functional signatures. In one or more embodiments, computing device may receive plurality of functional signatures and generate a graphical representation such as an X-Y chart of changes in functional signatures over time. In one or more embodiments, functional signature may be graphed relative to other individuals within a similar sex, age range, physical capability range, and the like. In one or more embodiments, functional signature may be graphed side by side and/or through an overlay wherein a patient may visually view their performance in comparison to others within a similar situation as user. In one or more embodiments, functional signatures of other patients and/or a calculated average may be displayed visually wherein a user may view their own functional signature in comparison to others.

Still referring to FIG. 2, conduct indicator 208 may include a dimensional element. As used in this disclosure a “dimensional element” is an element of datum denoting a relative measure of wellness of an individual. For example, and without limitation dimensional element may denote one or more dimensions associated with healthy living. In an embodiment dimensional element may include an occupational dimension. As used in this disclosure an “occupational dimension” is a dimension of wellness representing personal satisfaction and enrichment in an individual's life through work and/or occupation. For example, and without limitation, occupational dimension may denote that an individual's job is rewarding due to the contribution of personal values, interests, and/or beliefs that are shared among the job and the individual. In an embodiment dimensional element may include a physical dimension. As used in this disclosure a “physical dimension” is a dimension of wellness representing physical activity and/or nutrition. For example, and without limitation, physical dimension may include a dimension associated with eating whole grain foods and/or lean protein foods diet and/or nutrition, while concurrently discouraging the use of recreational drugs. As a further non-limiting example, physical dimension may include a dimension associated with regular exercise and/or enhanced physical strength. In an embodiment dimensional element may include a social dimension. As used in this disclosure a “social dimension” is a dimension of wellness representing an individual's contributions towards the environment and/or community. For example, and without limitation, social dimension may include a dimension associated with an individual's contributions towards the common welfare of the community and/or living in harmony with other.

In an embodiment and still referring to FIG. 2, dimensional element may include an intellectual dimension. As used in this disclosure a “intellectual dimension” is a dimension of wellness representing an individual's creative and/or mental activities. For example, and without limitation, intellectual dimension may include a dimension associated with an individual's abilities to identify potential problems and choose appropriate courses of action based on available information than to wait, worry, and contend with major concerns later. In an embodiment dimensional element may include a spiritual dimension. As used in this disclosure a “spiritual dimension” is a dimension of wellness representing an individual's search for meaning and/or purpose of existence. For example, and without limitation, spiritual dimension may include a dimension associated with an individual's understanding of the meaning for existence and/or the tolerance of other's meaning for existence. In an embodiment dimensional element may include an emotional dimension. As used in this disclosure an “emotional dimension” is a dimension of wellness representing an individual's awareness and/or acceptance of feelings. For example, and without limitation, emotional dimension may include a dimension associated with an individual's feelings related to a belief, philosophy, behavior, and the like thereof.

Still referring to FIG. 2, computing device 104 may receive conduct indicator 208 as a function of obtaining an exposure element. As used in this disclosure an “exposure element” is an element of datum representing contact and/or exposure associated with a lifestyle. For example, and without limitation exposure element may denote prolonged contact to radioactive material as a function of being a nuclear power plant technician. As a further non-limiting example exposure element may denote prolonged contact to illicit drugs as a function of being a recreational drug user. As a further non-limiting example, exposure element may denote prolonged contact to heavy metals in water as a function of having a surfing lifestyle. In an embodiment, and without limitation, exposure element may denote one or more exposures to toxins such as, but not limited to, persistent organic pollutants, polychlorinated bisphenols, hydrogen chlorides, benzenes, xylenes, toluenes, dioxins, heavy metals, radioactivity, and the like thereof. In another embodiment, exposure element may denote one or more epigenetic factors. As used in this disclosure an “epigenetic factor” is a factor denoting a likelihood of a change in gene activity and/or expression as a function of one or more external factors. For example, and without limitation, epigenetic factor may denote a high likelihood for a gene mutation as a function of a polyaromatic hydrocarbon. As a further non-limiting example, epigenetic factor may denote a high likelihood for reduced gene expression as a function of aluminum toxicity and/or poisoning.

In an embodiment, and still referring to FIG. 2, functional signature 204 may be obtained as a function of obtaining a salubrious reference. As used in this disclosure a “salubrious reference” is a guideline and/or recommendation representing an ideal health level of an individual. For example, and without limitation salubrious reference may include a guideline that a blood pressure should be 120/80 mmHg. As a further non-limiting example, salubrious reference may include a recommendation that a respiratory rate should be 14 breaths per minute. As a further non-limiting example, salubrious reference may denote that an individual should exercise for 30 minutes every other day. As a further non-limiting example, salubrious reference may denote that an individual should attend a religious gathering once a week. As a further non-limiting example, salubrious reference may denote that an individual should meditate twice a day for 10 minutes. As a further non-limiting example, salubrious reference may denote that an individual should have 5 or more chakras balanced during a particular time period, wherein a time period includes milliseconds, seconds, minutes, hours, days, weeks, months, years, and the like thereof. Salubrious reference may be obtained as a function of one or more informed advisors, wherein an informed advisor is described above in detail. Additionally or alternatively, salubrious reference may be obtained as a function of one or more functional advisors. As used in this disclosure a “functional advisor” is an individual capable of recommending and/or guiding an individual towards a more suited wellness state. For example, and without limitation, functional advisor may include one or more nutritionists, personal trainers, physical therapists, spiritual leaders, religious leaders, massage therapists, spiritual therapists, reiki masters, acupuncturists, life coaches, priests, philosophers, theologists, yoga instructors, wellness instructors, teachers, and the like thereof. In an embodiment, salubrious reference may include recommendations from one or more medical sources such as peer reviews, informed advisor associations, medical websites, medical textbooks, religious books, prophecies, spiritual texts, and the like thereof. In one or more embodiments, conduct parameters may be generated as a function of salubrious reference. In one or more embodiments, each conduct parameter may be associated with each salubrious reference. In one or more embodiments, conduct parameters may include guidelines of ideal health as described above. In one or more embodiments, computing device may generate and/or select conduct parameters based on salubrious references.

Still referring to FIG. 2, computing device 104 identifies functional signature 204 as function of conduct indicator 208 and respiratory parameter 112 using a functional machine-learning model 212. As used in this disclosure a “functional machine-learning model” is a machine-learning model that identifies a functional signature output given respiratory parameters and conduct indicators as inputs; this is in contrast to a non-machine learning software program where the commands to be executed are determined in advance by a user and written in a programming language. Functional machine-learning model may include one or more functional machine-learning processes such as supervised, unsupervised, or reinforcement machine-learning processes that computing device 104 and/or a remote device may or may not use in the determination of functional signature 204, wherein a remote device is an external device to computing device 104 as described above in detail. A functional machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 2, computing device 104 may train functional machine-learning process as a function of a functional training set. As used in this disclosure a “functional training set” is a training set that correlates a respiratory parameter and a conduct indicator to a functional signature. For example, and without limitation, a respiratory parameter of reduced tidal volume and a conduct indicator associated with smoking cigarettes for 20 years may relate to a functional signature of a relatively “unhealthy” wellness state. The functional training set may be received as a function of user-entered valuations of respiratory parameters, conduct indicators, and/or functional signatures. Computing device 104 may receive functional training set by receiving correlations of respiratory parameters and/or conduct indicators that were previously received and/or determined during a previous iteration of determining functional signatures. The functional training set may be received by one or more remote devices that at least correlate a respiratory parameter and conduct indicator to a functional signature, wherein a remote device is an external device to computing device 104, as described above. Functional training set may be received in the form of one or more user-entered correlations of a respiratory parameter and/or conduct indicator to a functional signature. Additionally or alternatively, a user may include, without limitation, an informed advisor and/or a functional advisor entering correlations of respiratory parameters and/or conduct indicators to functional signatures, wherein informed advisors and/or functional advisors may include, without limitation, physicians, nutritionists, therapists, spiritual leaders, and the like thereof as described above in detail.

Still referring to FIG. 2, computing device 104 may receive functional machine-learning model 212 from a remote device that utilizes one or more functional machine learning processes, wherein remote device is described above in detail. For example, and without limitation, remote device may include a computing device, external device, processor, and the like thereof. Remote device may perform the functional machine-learning process using the functional training set to generate functional signature 204 and transmit the output to computing device 104. Remote device may transmit a signal, bit, datum, or parameter to computing device 104 that at least relates to functional signature 204. Additionally or alternatively, the remote device may provide an updated machine-learning model. For example, and without limitation, an updated machine-learning model may be comprised of a firmware update, a software update, a functional machine-learning process correction, and the like thereof. As a non-limiting example a software update may incorporate a new respiratory parameter that relates to a modified conduct indicator. Additionally or alternatively, the updated machine learning model may be transmitted to the remote device, wherein the remote device may replace the functional machine-learning model with the updated machine-learning model and determine the physiological as a function of the conduct indicator using the updated machine-learning model. The updated machine-learning model may be transmitted by the remote device and received by computing device 104 as a software update, firmware update, or corrected functional machine-learning model. For example, and without limitation a functional machine-learning model 212 may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, functional machine-learning model 212 may identify functional signature 204 as a function of one or more classifiers, wherein a classifier is described above in detail.

Still referring to FIG. 2, computing device 104 may identify functional signature 204 by producing an indicator index as a function of conduct indicator 208. As used in this disclosure an “indicator index” is a measurable value associated with a conduct indicator. For example and without limitation, an indicator index may be 20 for a conduct indicator associated with meditating 3 times a day for 5 minutes. As a further non-limiting example, an indicator index may be 73 for a conduct indicator associated with a sedentary lifestyle comprising sitting down for 12 hours a day. In one or more embodiments indicator index may be generated by comparing conduct indicator 208 to conduct parameters. In one or more embodiments, indicator index may be generated by a comparison of conduct indicator 208 to conduct parameters wherein adherence to one or more conduct parameters may signify a particular score. A “conduct parameter” for the purposes of this disclosure is a set of limits indicating ideal limits to one or more elements within conduct indicator 208. For example, and without limitation, conduct parameter may include ideal eating habits, ideal exercise, ideal meditation, ideal sleep patterns and/or any other elements within conduct parameter. In an embodiments, conduct parameter may indicate ideal cholesterol levels, ideal weight levels, ideal happiness levels and the like. In an embodiment, computing device 104 may be configured to compare conduct indicator 208 to one or more conduct parameters and generated indicator index as a result. In one or more embodiments, computing device 104 may convert conduct indicator and conduct parameter to numerical values and compare the numerical values to generate indicator index. In one or more embodiments, numerical values may be generated based on predetermined values in a lookup table, linear expressions and the like. In one or more embodiments, conduct parameters may be generated by a medical professional, such a doctor, a physician, an individual holding themselves out as proficient in nutrients and/or medicine and the like. In one or more embodiments, conduct parameter may be generated using a WebCrawler wherein the web crawler is configured to retrieve conduct parameters from one or more medical related websites of healthy individuals. In one or more embodiments, computing device 104 may generate averages to be compared to and the like. In one or more embodiments, computing device 104 may select at least one conduct parameter from a plurality of conduct parameters. In one or more embodiments, plurality of conduct parameters may be stored and/or located on a database such as any database as described in this disclosure. In one or more embodiments, computing device may be configured to select at least one conduct parameter as a function of user physiological data. “User physiological data” for the purposes of this disclosure is information associated with the physical characteristics of user. For example, and without limitation, user physiological data may include but is not limited to, age, gender, height, weight, a particular diagnosis, a particular medication taken, a particular ethnic background, dietary restrictions, how active the user may be and the like. In one or more embodiments, conduct indicator may be compared to conduct parameters that are closely related to user. For example, and without limitation, a user who is male be compared to conduct parameters associated with males. Similarly, a user with diabetes may be compared to conduct parameters associated with diabetes. In an embodiment, a user may be compared to ideal conduct indicators within the range of one with similar age, sex and/or physical capabilities. In one or more embodiments, computing device 104 may receive user physiological data from a user and select conduct parameters associated with user physiological data. In one or more embodiments, conduct parameters may be grouped by age, gender, physical abilities and the like wherein computing device 104 may select a grouping most closely associated with user. In one or more embodiments, computing device 104 may use a classifier such as any classifier as described in this disclosure to select conduct parameters from a plurality of conduct parameters wherein indicator index may be generated as a result. In an embodiment, computing device may produce a weighted index as a function of the indicator index and pulmonary dysfunction. As used in this disclosure a “weighted index” is a weighted value associated with conduct indicator and pulmonary dysfunction. For example, and without limitation, a conduct indicator of a lifestyle of smoking tobacco for 30 years may relate to a value of 18, wherein a pulmonary dysfunction for COPD may weight and/or alter the value to adjust to 73.

Still referring to FIG. 2, computing device 104 may identify functional signature 204 as a function of determining a root cause. As used in this disclosure a “root cause” is a source of origination of a conduct indicator. For example, and without limitation, root cause may denote that an individual has a sedentary lifestyle as a function of watching television. As a further non-limiting root cause may denote that an individual started smoking as a function of a lack of religious guidance and/or spiritual teaching. As a further non-limiting example, root cause may denote that an individual has emotional instability as a function of one or more traumatic experiences and/or psychological traumas. Additionally or alternatively, computing device 104 may determine a habit as a function of conduct indicator 208. As used in this disclosure a “habit” is a tendency and/or regularly practiced behavior that an individual performs. For example, and without limitation a habit may include swearing, trichotillomania, picking an individual's nose, smoking cigarettes, biting fingernails, drinking coffee, drinking tea, hair picking, watching television, eating fast food, alcohol, emotional shopping, social media use, drinking soda, eating chocolate, humming, sleeping-in, lying, procrastinating, being unfriendly, and the like thereof.

Still referring to FIG. 2, computing device 104 is configured to generate a functional program 216 as a function of functional signature 204. As used in this disclosure a “functional program” is a program and/or instruction set to alter an individual's lifestyle to affect respiratory parameter 112 and/or functional signature 204. A functional program may provide instruction relating to one or more areas of a user's life, including but not limited to, physical fitness, stress management, meditation, spirituality, religion, energy healing, professional endeavors, personal endeavors, body, mind, health, finances, recreation, romance, personal development, and the like. For example, and without limitation, functional program 216 may include a program that instructs an individual to perform 10 minutes of strenuous exercise every day for 5 weeks. As a further non-limiting example, functional program 216 may include a program that instructs an individual to meditate for 1 minute every other week. As a further non-limiting example, functional program 216 may instruct an individual to go on a hike for 2 hours once a week. Additionally or alternatively, functional program 216 may include a nourishment program 144, wherein nourishment program 144 is described above in detail, in reference to FIG. 1. For example, and without limitation, functional program 216 may instruct an individual to consume a paleo diet. In an embodiment and without limitation, functional program 216 may include one or more instructions such as, but not limited to a first instruction to exercise and a second instruction of a nourishment program. In one or more embodiments nourishment program may be transmitted periodically to a remote device associated with user. In one or more embodiments, remote device may include but is not limited to, a wearable smart device, a smart phone, a laptop and the like. In one or more embodiments, nourishment program may be transmitted periodically to remind user of adherence to nourishment program. In one or more embodiments, nourishment program may be transmitted at a particular times of the day such as before meals, at sunset, at a particular time and the like. In one or more embodiments, nourishment program may be transmitted periodically to ensure compliance and/or adherence with nourishment program. Computing device 104 may functional program 216 as a function of functional signature 204 using a program machine-learning model. As used in this disclosure a “program machine-learning model” is a machine-learning model that produces a functional program output given functional signatures as inputs; this is in contrast to a non-machine learning software program where the commands to be executed are determined in advance by a user and written in a programming language. Program machine-learning model may include one or more program machine-learning processes such as supervised, unsupervised, or reinforcement machine-learning processes that computing device 104 and/or a remote device may or may not use in the determination of functional program 216, wherein a remote device is an external device to computing device 104 as described above in detail. A program machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

With continued reference to FIG. 2, computing device may generate predicted functional signature as a function of an adherence to nourishment program. A “Predicted functional signature” for the purposes of this disclosure is a calculated future functional signature based on actions made by the user in association to their health. For example, and without limitation, predicted functional signature may increase or decrease in comparison to functional signature based on foods consumed by the user, new exercises taken and the like. In one or more embodiments, computing device 104 may determine an adherence to nourishment program may receiving information associated with a user's implementation to nourishment program and/or planned implementation to nourishment program. In one or more embodiments, computing device 104 may receive information from a user indicating that the user will adhere to several steps outlined in nourishment program wherein computing device 104 may generate predicted functional signature as a result. In one or more embodiments, computing device 104 may receive in the future, a user's habits, adherences to nourishment program and the like wherein computing device may generate predicted functional signature. In one or more embodiments, computing device 104 may receive one or more inputs from a user indicating that a user will implement one or more elements within nourishment program wherein computing device 104 may generate predicted functional signature as a result. In one or more embodiments, predicted functional signature may be generated in any way as described in this disclosure. In one or more embodiments, elements within nourishment program may be associated with values and/or numerical values wherein adherence to each step or element may indicate a predicted functional signature. In one or more embodiments, numerical values associated with elements within nourishment program may be used to offset functional signature wherein predicated functional signature may be generated as a function of the offset. In one or more embodiments, one or more machine learning models as described in this disclosure may be used to generate predicted functional signature wherein inputs may include inputs such as adherence to one or more steps within nourishment program and outputs may include predicted functional signatures.

With continued reference to FIG. 2, computing device 104 may be configured to select at least one nourishment coach as a function of nourishment program. A “nourishment coach” for the purposes of this disclosure is a person or system who is tasked with assisting a user with implementing nourishment program. For example, and without limitation, nourishment coach may include a medical professional, nutritionist and/or a similar individual tasked with communicating with user in order to ensure adherence to nourishment program. In one or more embodiments, nourishment coach may be tasked with assisting user adhere to current nourishment program. In one or more embodiments, nourishment coach may further assist user in creating more dietary restrictions in order to ensure that the user's health improves. In one or more embodiments, a diabase such as any database as described in this disclosure may contain a plurality of nourishment coaches, wherein nourishment coaches may be selected based on user physiological data as described above, based on a user's location based on outputs of nourishment program and the like. In one or more embodiments, nourishment coach may include a chatbot system connected to a large language model. A “chatbot system” for the purposes of this disclosure is a program configured to simulate human interaction with a user in order to receive or convey information. In some cases, chatbot system may be configured to receive nourishment program and/or elements thereof and any other data as described in this disclosure and output data in a communicative format. In one or more embodiments, chatbot system may be configured to simulate human interaction wherein chatbot may communicate in a natural language format and simulate human interaction. In one or more embodiments, chatbot system may be configured to simulate human interaction in a variety of languages based on the preferences of a user. In one or more embodiments, while data processing and/or information received may be in a particular language, chatbot may be configured to translate data based on the preferences of the user. In one or more embodiments, chatbot system may utilize a large language model in order to interact with user and process information. In one or more embodiments, nourishment coach may include a large language model configured to ensure adherence of user to nourishment program.

Still referring to FIG. 2, nourishment coach may include a large language model (LLM). A “large language model,” as used herein, is a deep learning data structure that can recognize, summarize, translate, predict and/or generate text and other content based on knowledge gained from massive datasets. Large language models may be trained on large sets of data. Training sets may be drawn from diverse sets of data such as, as non-limiting examples, novels, blog posts, articles, emails, unstructured data, electronic records, and the like. In some embodiments, training sets may include a variety of subject matters, such as, as nonlimiting examples, medical report documents, electronic health records, entity documents, business documents, inventory documentation, emails, user communications, advertising documents, newspaper articles, and the like. In some embodiments, training sets of an LLM may include information from one or more public or private databases. As a non-limiting example, training sets may include databases associated with an entity. In some embodiments, training sets may include portions of documents associated with the electronic records 112 correlated to examples of outputs. In an embodiment, an LLM may include one or more architectures based on capability requirements of an LLM. Exemplary architectures may include, without limitation, GPT (Generative Pretrained Transformer), BERT (Bidirectional Encoder Representations from Transformers), T5 (Text-To-Text Transfer Transformer), and the like. Architecture choice may depend on a needed capability such generative, contextual, or other specific capabilities.

With continued reference to FIG. 2, in some embodiments, an LLM may be generally trained. As used in this disclosure, a “generally trained” LLM is an LLM that is trained on a general training set comprising a variety of subject matters, data sets, and fields. In some embodiments, an LLM may be initially generally trained. Additionally, or alternatively, an LLM may be specifically trained. As used in this disclosure, a “specifically trained” LLM is an LLM that is trained on a specific training set, wherein the specific training set includes data including specific correlations for the LLM to learn. As a non-limiting example, an LLM may be generally trained on a general training set, then specifically trained on a specific training set. In an embodiment, specific training of an LLM may be performed using a supervised machine learning process. In some embodiments, generally training an LLM may be performed using an unsupervised machine learning process. As a non-limiting example, specific training set may include information from a database. As a non-limiting example, specific training set may include text related to the users such as user specific data for electronic records correlated to examples of outputs. In an embodiment, training one or more machine learning models may include setting the parameters of the one or more models (weights and biases) either randomly or using a pretrained model. Generally training one or more machine learning models on a large corpus of text data can provide a starting point for fine-tuning on a specific task. A model such as an LLM may learn by adjusting its parameters during the training process to minimize a defined loss function, which measures the difference between predicted outputs and ground truth. Once a model has been generally trained, the model may then be specifically trained to fine-tune the pretrained model on task-specific data to adapt it to the target task. Fine-tuning may involve training a model with task-specific training data, adjusting the model's weights to optimize performance for the particular task. In some cases, this may include optimizing the model's performance by fine-tuning hyperparameters such as learning rate, batch size, and regularization. Hyperparameter tuning may help in achieving the best performance and convergence during training. In an embodiment, fine-tuning a pretrained model such as an LLM may include fine-tuning the pretrained model using Low-Rank Adaptation (LoRA). As used in this disclosure, “Low-Rank Adaptation” is a training technique for large language models that modifies a subset of parameters in the model. Low-Rank Adaptation may be configured to make the training process more computationally efficient by avoiding a need to train an entire model from scratch. In an exemplary embodiment, a subset of parameters that are updated may include parameters that are associated with a specific task or domain.

With continued reference to FIG. 2, in some embodiments an LLM may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. An LLM may include a text prediction based algorithm configured to receive an article and apply a probability distribution to the words already typed in a sentence to work out the most likely word to come next in augmented articles. For example, if some words that have already been typed are “Nice to meet”, then it may be highly likely that the word “you” will come next. An LLM may output such predictions by ranking words by likelihood or a prompt parameter. For the example given above, an LLM may score “you” as the most likely, “your” as the next most likely, “his” or “her” next, and the like. An LLM may include an encoder component and a decoder component.

Still referring to FIG. 2, an LLM may include a transformer architecture. In some embodiments, encoder component of an LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 2, an LLM and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 2, attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, an LLM may predict the next word by searching for a set of positions in a source sentence where the most relevant information is concentrated. An LLM may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 2, attention mechanism may include, without limitation, generalized attention self-attention, multi-head attention, additive attention, global attention, and the like. In generalized attention, when a sequence of words or an image is fed to an LLM, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, an LLM may pick up particular parts at different positions in the input sequence and over time compute an initial composition of the output sequence. In multi-head attention, an LLM may include a transformer model of an attention mechanism. Attention mechanisms, as described above, may provide context for any position in the input sequence. For example, if the input data is a natural language sentence, the transformer does not have to process one word at a time. In multi-head attention, computations by an LLM may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), an LLM may make use of attention alignment scores based on a number of factors. Alignment scores may be calculated at different points in a neural network, and/or at different stages represented by discrete neural networks. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, an LLM may either attend to all source words or predict the target sentence, thereby attending to a smaller subset of words.

With continued reference to FIG. 2, multi-headed attention in encoder may apply a specific attention mechanism called self-attention. Self-attention allows models such as an LLM or components thereof to associate each word in the input, to other words. As a non-limiting example, an LLM may learn to associate the word “you”, with “how” and “are”. It's also possible that an LLM learns that words structured in this pattern are typically a question and to respond appropriately. In some embodiments, to achieve self-attention, input may be fed into three distinct fully connected neural network layers to create query, key, and value vectors. Query, key, and value vectors may be fed through a linear layer; then, the query and key vectors may be multiplied using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

Still referencing FIG. 2, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 2, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

Continuing to refer to FIG. 2, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With further reference to FIG. 2, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 2, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “0s” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.”

Still referring to FIG. 2, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 2, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

Still referring to FIG. 2, decoder may take this output and add it to the decoder inputs. Decoder may continue decoding until a token is predicted. Decoder may stop decoding once it predicts an end token.

Continuing to refer to FIG. 2, in some embodiment, decoder may be stacked N layers high, with each layer taking in inputs from the encoder and layers before it. Stacking layers may allow an LLM to learn to extract and focus on different combinations of attention from its attention heads.

With continued reference to FIG. 2, an LLM may receive an input. Input may include a string of one or more characters. Inputs may additionally include unstructured data. For example, input may include one or more words, a sentence, a paragraph, a thought, a query, and the like. A “query” for the purposes of the disclosure is a string of characters that poses a question. In some embodiments, input may be received from a user device. User device may be any computing device that is used by a user. As non-limiting examples, user device may include desktops, laptops, smartphones, tablets, and the like. In some embodiments, input may include any set of data associated with nourishment program wherein LLM may output advice associated with nourishment program, additional restrictions and the like.

With continued reference to FIG. 1, an LLM may generate at least one annotation as an output. At least one annotation may be any annotation as described herein. In some embodiments, an LLM may include multiple sets of transformer architecture as described above. Output may include a textual output. A “textual output,” for the purposes of this disclosure is an output comprising a string of one or more characters. Textual output may include, for example, a plurality of annotations for unstructured data. In some embodiments, textual output may include a phrase or sentence identifying the status of a user query. In some embodiments, textual output may include a sentence or plurality of sentences describing a response to a user query. As a non-limiting example, this may include restrictions, timing, advice, dangers, benefits, and the like.

Still referring to FIG. 2, computing device 104 may train program machine-learning process as a function of a program training set. As used in this disclosure a “program training set” is a training set that correlates a functional signature to a functional program. For example, and without limitation, a functional signature of a habit of being exposed to radioactivity may relate to a functional program of a reduced exposure to radioactivity, exercise for 30 minutes to aid in eliminating the toxin, and increased meditation to reduce inflammation. The program training set may be received as a function of user-entered valuations of functional signatures, and/or functional programs. Computing device 104 may receive program training set by receiving correlations of functional signatures that were previously received and/or determined during a previous iteration of determining functional programs. The program training set may be received by one or more remote devices that at least correlate a functional signature to a functional program, wherein a remote device is an external device to computing device 104, as described above. Program training set may be received in the form of one or more user-entered correlations of a functional signature to a functional program. Additionally or alternatively, a user may include, without limitation, an informed advisor and/or a functional advisor entering correlations of functional signatures to functional programs, wherein informed advisors and/or functional advisors may include, without limitation, physicians, nutritionists, therapists, spiritual leaders, and the like thereof as described above in detail.

Still referring to FIG. 2, computing device 104 may receive program machine-learning model from a remote device that utilizes one or more program machine learning processes, wherein remote device is described above in detail. For example, and without limitation, remote device may include a computing device, external device, processor, and the like thereof. Remote device may perform the program machine-learning process using the program training set to generate functional program 216 and transmit the output to computing device 104. Remote device may transmit a signal, bit, datum, or parameter to computing device 104 that at least relates to functional program 216. Additionally or alternatively, the remote device may provide an updated machine-learning model. For example, and without limitation, an updated machine-learning model may be comprised of a firmware update, a software update, a program machine-learning process correction, and the like thereof. As a non-limiting example a software update may incorporate a new functional signature that relates to a modified functional program. Additionally or alternatively, the updated machine learning model may be transmitted to the remote device, wherein the remote device may replace the program machine-learning model with the updated machine-learning model and determine the functional program as a function of the functional signature using the updated machine-learning model. The updated machine-learning model may be transmitted by the remote device and received by computing device 104 as a software update, firmware update, or corrected program machine-learning model. For example, and without limitation a program machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, program machine-learning model may identify functional program 216 as a function of one or more classifiers, wherein a classifier is described above in detail.

Still referring to FIG. 2, computing device 104 may generate functional program 216 as a function of determining a holistic prospect. As used in this disclosure a “holistic prospect” is a potential adjustment to an individual's functional signature. For example, and without limitation, holistic project may denote that a potential adjustment may include adjusting the amount of exercise and/or strenuous activity performed by the individual. As a further non-limiting example, holistic project may denote that a potential adjustment may include adjusting the amount of religious guidance that an individual receives. As a further non-limiting example, holistic project may denote that a potential adjustment may include adjusting the amount of chakra flow of an individual. As a further non-limiting example, holistic project may denote that a potential adjustment may include adjusting the number of social interactions that an individual experiences each day. As a further non-limiting example, holistic prospect may include a potential adjustment to a nourishment program through the alteration of one or more edibles and/or supplementation of a nourishment program. In an embodiment, holistic prospect may include one or more supplements. For example, and without limitation, a supplement may include vitamin E, linoleic acid, lipoic acid, inositol, magnesium, biotin, progestin, vitamin D, and the like thereof.

Still referring to FIG. 2, computing device 104 generate functional program 216 as a function of a pulmonary functional goal. As used in this disclosure an “pulmonary functional goal” is a predicted goal and/or purposeful plan to modify functional signature 204 and/or respiratory parameter 112. As a non-limiting example, pulmonary functional goal may include a treatment goal. As used in this disclosure a “treatment goal” is a pulmonary functional goal that is designed to at least reverse and/or eliminate functional signature 204, respiratory parameter 112, and/or pulmonary dysfunction. As a non-limiting example, a treatment goal may include reversing the effects of COPD as a function of exercise, diet, and/or supplementation. As a further non-limiting example, a treatment goal includes reversing SARS-CoV-2 as a function of recommending the supplement N-acetyl cysteine, recommending edibles such as apples, berries, tomatoes, celery, onions, sauerkraut, kombucha, and the like thereof, recommending a meditation schedule of once per day for 20 minutes, and/or recommending 25 minutes of exercise every other day. Pulmonary functional goal may include a prevention goal. As used in this disclosure a “prevention goal” is a pulmonary functional goal that is designed to at least prevent and/or avert functional signature 204, respiratory parameter 112, and/or pulmonary dysfunction. As a non-limiting example, a prevention goal may include preventing the development of the asthma as a function of hiking 2 miles per day and/or recommending a nourishment program of a low-carb diet. Pulmonary functional goal may include a mitigation goal. As used in this disclosure a “mitigation goal” is a functional goal that is designed to reduce the symptoms and/or effects of a pulmonary dysfunction. For example, and without limitation, mitigation goal may include reducing the effects of lung cancer as a function of recommending magnesium and/or zinc supplements and/or recommending enhanced chakra flow of an individual's body. Additionally or alternatively, pulmonary functional goal may include one or more goals associated with gene therapy to alter and/or mutate an individual's epigenetic factors.

Still referring to FIG. 2, computing device 104 may generate functional program 216 as a function of functional signature 204 and pulmonary functional goal using a goal machine-learning model. As used in this disclosure a “goal machine-learning model” is a machine-learning model to produce a functional program output given functional signatures and/or pulmonary functional goals as inputs; this is in contrast to a non-machine learning software program where the commands to be executed are determined in advance by a user and written in a programming language. Goal machine-learning model may include one or more goal machine-learning processes such as supervised, unsupervised, or reinforcement machine-learning processes that computing device 104 and/or a remote device may or may not use in the development of functional program 216. Goal machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 2, computing device 104 may train goal machine-learning process as a function of a goal training set. As used in this disclosure a “goal training set” is a training set that correlates a pulmonary functional goal to a functional signature. The goal training set may be received as a function of user-entered functional signatures, pulmonary functional goals, and/or functional programs. For example, and without limitation, a pulmonary functional goal of treating emphysema may correlate to a functional signature of physical activity and/or a vegan diet. Computing device 104 may receive goal training by receiving correlations of pulmonary functional goals and/or functional signatures that were previously received and/or determined during a previous iteration of generating functional programs. The goal training set may be received by one or more remote devices that at least correlate a pulmonary functional goal and/or functional signature to a functional program, wherein a remote device is an external device to computing device 104, as described above. Goal training set may be received in the form of one or more user-entered correlations of a pulmonary functional goal and/or functional signature to a functional program. Additionally or alternatively, a user may include, without limitation, an informed advisor and/or a functional advisor entering correlations of functional signatures and/or pulmonary appraisals to functional programs, wherein informed advisors and/or functional advisors may include, without limitation, physicians, nutritionists, therapists, spiritual leaders, and the like thereof as described above in detail.

Still referring to FIG. 2, computing device 104 may receive goal machine-learning model from the remote device that utilizes one or more goal machine learning processes, wherein a remote device is described above in detail. For example, and without limitation, a remote device may include a computing device, external device, processor, and the like thereof. The remote device may perform the goal machine-learning process using the goal training set to develop functional program 216 and transmit the output to computing device 104. The remote device may transmit a signal, bit, datum, or parameter to computing device 104 that at least relates to functional program 216. Additionally or alternatively, the remote device may provide an updated machine-learning model. For example, and without limitation, an updated machine-learning model may be comprised of a firmware update, a software update, a goal machine-learning process correction, and the like thereof. As a non-limiting example a software update may incorporate a new pulmonary functional goal that relates to a modified functional signature. Additionally or alternatively, the updated machine learning model may be transmitted to the remote device, wherein the remote device may replace the goal machine-learning model with the updated machine-learning model and develop the functional program as a function of the pulmonary functional goal using the updated machine-learning model. The updated machine-learning model may be transmitted by the remote device and received by computing device 104 as a software update, firmware update, or corrected goal machine-learning model. For example, and without limitation goal machine-learning model may utilize a neural net machine-learning process, wherein the updated machine-learning model may incorporate decision tree machine-learning processes.

Now referring to FIG. 3, an exemplary embodiment 300 of a representative diagram of respiratory parameter 112 according to an embodiment of the invention is illustrated. Respiratory parameter 112 may include one or more respiratory measurements according to a breathing cycle 304. As used in this disclosure “breathing cycle” is the movement of air during both inhalation and exhalation, wherein inhalation is represented as an increase along a y-axis representing volume in mL/kg and exhalation is represented as a decreases along a x-axis representing time. Breathing cycle 304 may represent both regulatory breathing, wherein the individual is not participating in any strenuous respiratory activities and/or strenuous breathing, wherein the individual is inhaling air in an attempt to maximize the inhalation and exhaling the maximum amount of air that was inhaled. Respiratory parameter 112 may include an inspiratory reserve volume (IRV) 308 as a function of breathing cycle 304. As used in this disclosure “inspiratory reserve volume (IRV)” is the additional amount of air that can be inhaled after a normal inhalation. As a non-limiting example IRV 308 may include an individual that may normally inhale 2 L of air, wherein an additional 1.5 L of air may be additionally inhaled. Respiratory parameter 112 may include a tidal volume (TV) 312 as a function of breathing cycle 304. As used in this disclosure “tidal volume (TV)” is the amount of air that is inspired and expired during a normal breath. As a non-limiting example, TV 312 may include an individual that normally inhales and exhales 1 L of air. Respiratory parameter 112 may include calculating an expiratory reserve volume (ERV) 316 as a function of breathing cycle 304. As used in this disclosure “expiratory reserve volume (ERV)” is the additional amount of air that can be exhaled after a normal exhalation. As a non-limiting example, ERV 316 may include an individual that may normally exhale 1 L of air, wherein an additional 2.5 L of air may be additionally exhaled. Respiratory parameter 112 may include a residual volume (RV) 320 as a function of breathing cycle 304. As used in this disclosure “residual volume (RV)” is the additional amount of air that is left after ERV is exhaled. As a non-limiting example, RV 320 may include an individual that has exhaled 2.5 L of ERV, wherein 1.25 L of air remains in the lungs of the individual.

Still referring to FIG. 3, respiratory parameter 112 may include inspiratory capacity (IC) 324 as a function of breathing cycle 304. As used in this disclosure “inspiratory capacity (IC)” is the amount of air that may be inhaled after the end of a normal expiration. As a non-limiting example IC 324 may include a total volume of 3.25 L that may be inhaled after an individual has normally exhaled. Respiratory parameter 112 may include functional residual capacity (FRC) 328. As used in this disclosure “functional residual capacity (FRC)” is the amount of additional air that can be exhaled after a normal exhalation. As a non-limiting example FRC 328 may include a total volume of 2.75 L that may be exhaled after an individual has normally inhaled. Respiratory parameter 112 may include vital capacity (VC) 332. As used in this disclosure “vital capacity (VC)” is the maximum amount of air that can be inhaled or exhaled during a respiratory cycle. As a non-limiting example VC 332 may the sum of ERV, TV, and IRV to determine a maximum amount of air that may be inhaled and/or exhaled by an individual. Respiratory parameter 112 may include a total lung capacity (TLC) 336. As used in this disclosure “total lung capacity (TLC)” is the total amount of air that an individual's lung may hold. As a non-limiting example TLC 336 may the sum of RV, ERV, TV, and IRV to determine a total amount of air that an individual's lung may hold.

Now referring to FIG. 4, an exemplary embodiment 400 of an edible directory 132 according to an embodiment of the invention is illustrated. Edible directory 132 may be implemented, without limitation, as a relational databank, a key-value retrieval databank such as a NOSQL databank, or any other format or structure for use as a databank that a person skilled in the art would recognize as suitable upon review of the entirety of this disclosure. Edible directory 132 may alternatively or additionally be implemented using a distributed data storage protocol and/or data structure, such as a distributed hash table or the like. Edible directory 132 may include a plurality of data entries and/or records as described above. Data entries in a databank may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a databank may store, retrieve, organize, and/or reflect data and/or records as used herein, as well as categories and/or populations of data consistently with this disclosure. Edible directory 132 may include a carbohydrate tableset 404. Carbohydrate tableset 404 may relate to a nourishment composition of an edible with respect to the quantity and/or type of carbohydrates in the edible. As a non-limiting example, carbohydrate tableset 404 may include monosaccharides, disaccharides, oligosaccharides, polysaccharides, and the like thereof. Edible directory 132 may include a fat tableset 408. Fat tableset 408 may relate to a nourishment composition of an edible with respect to the quantity and/or type of esterified fatty acids in the edible. Fat tableset 408 may include, without limitation, triglycerides, monoglycerides, diglycerides, phospholipids, sterols, waxes, and free fatty acids. Edible directory 132 may include a fiber tableset 412. Fiber tableset 412 may relate to a nourishment composition of an edible with respect to the quantity and/or type of fiber in the edible. As a non-limiting example, fiber tableset 412 may include soluble fiber, such as beta-glucans, raw guar gum, psyllium, inulin, and the like thereof as well as insoluble fiber, such as wheat bran, cellulose, lignin, and the like thereof. Edible directory 132 may include a mineral tableset 416. Mineral tableset 416 may relate to a nourishment composition of an edible with respect to the quantity and/or type of minerals in the edible. As a non-limiting example, mineral tableset 416 may include calcium, phosphorous, magnesium, sodium, potassium, chloride, sulfur, iron, manganese, copper, iodine, zing, cobalt, fluoride, selenium, and the like thereof. Edible directory 132 may include a protein tableset 420. Protein tableset 420 may relate to a nourishment composition of an edible with respect to the quantity and/or type of proteins in the edible. As a non-limiting example, protein tableset 420 may include amino acids combinations, wherein amino acids may include, without limitation, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and the like thereof. Edible directory 132 may include a vitamin tableset 424. Vitamin tableset 424 may relate to a nourishment composition of an edible with respect to the quantity and/or type of vitamins in the edible. As a non-limiting example, vitamin tableset 424 may include vitamin A, vitamin B₁, vitamin B₂, vitamin B₃, vitamin B₅, vitamin B₆, vitamin B₇, vitamin B₉, vitamin B₁₂, vitamin C, vitamin D, vitamin E, vitamin K, and the like thereof.

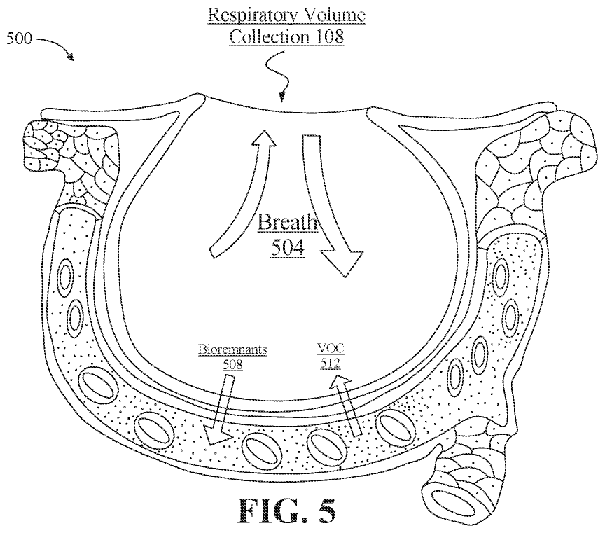

Now referring to FIG. 5 an exemplary embodiment 500 of biomarkers that may be received from respiratory volume collection 108 is illustrated. Respiratory volume collection 108 may collect a breath 504. As used in this disclosure “breath” is air that is taken into and/or expelled from the lungs, wherein the air interacts with one or more alveolus of the lung. Breath 504 may include one or more chemical elements such as nitrogen, oxygen, argon, carbon dioxide, neon, helium, and/or hydrogen. As a non-limiting example, breath 504 may be comprised of 78% of nitrogen, 20.95% of oxygen, and 1.05% of neon. As a further non-limiting example, breath 504 may be comprised of 78% nitrogen, 16% oxygen, 1% argon, and 5% carbon dioxide. Breath 504 may contain a bioremnant 508. As used in this disclosure “bioremnant” is a biological component that originates from an individual's body that represents the status of an individual's respiratory system. As a non-limiting example bioremnant 508 may include a biological component such as a Lung function, Alpha1-antitrypsin (AAT), angiogenic growth factor, brain natriuretic peptide (BNP), calprotectin, CF-specific serum proteomic signature, chromagranim A (CgA), copeptin, C-reactive protein (CRP), IgE, Nitric oxide, osteoprotegerin, parathyroid hormone, serum amyloid A, surfactant proteins, and the like thereof. Breath 504 may include a volatile organic compound (VOC) 512. As used in this disclosure “volatile organic compound (VOC)” is an organic compound that has a high vapor pressure that allows the organic compound to evaporate and/or sublime at room temperature. VOC 512 may include one or more biologically generated VOCs, wherein biologically generated VOCs may include, without limitation, isoprene, terpenes, pinene isomers, sesquiterpenes, methanol, acetone, and the like thereof. As a non-limiting example VOC 512 may include benzene, ethylene glycol, formaldehyde, methylene chloride, tetrachloroethylene, toluene, xylene, 1,3-butadiene, and the like thereof.

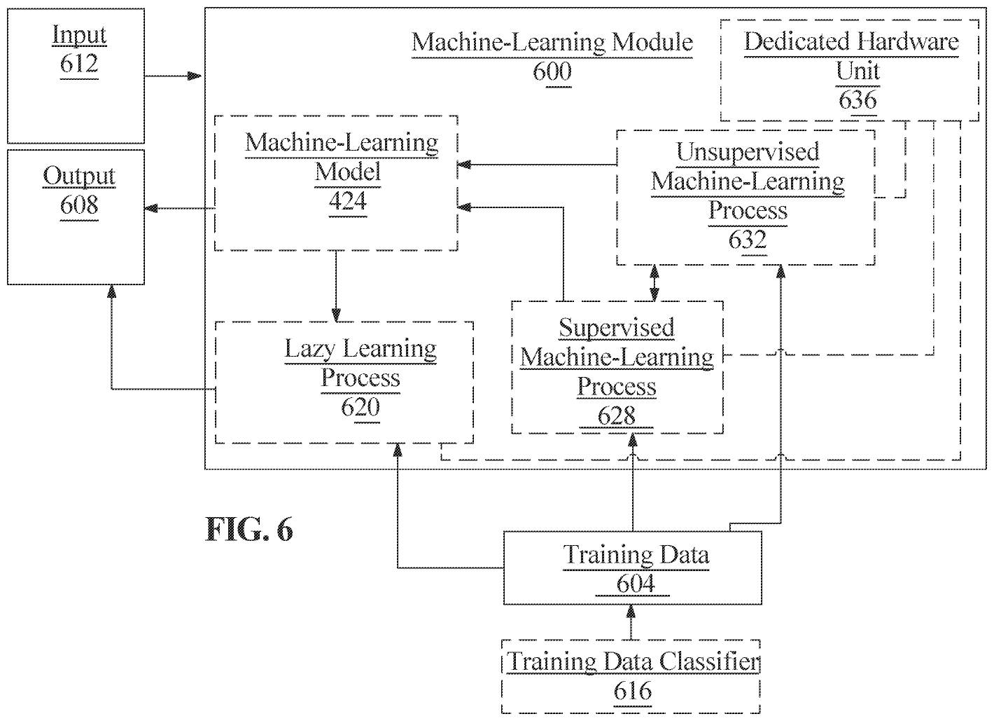

Referring now to FIG. 6, an exemplary embodiment of a machine-learning module 600 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 604 to generate an algorithm instantiated in hardware or software logic, data structures, and/or functions that will be performed by a computing device/module to produce outputs 608 given data provided as inputs 612; this is in contrast to a non-machine learning software program where the commands to be executed are determined in advance by a user and written in a programming language.

Still referring to FIG. 6, “training data,” as used herein, is data containing correlations that a machine-learning process may use to model relationships between two or more categories of data elements. For instance, and without limitation, training data 604 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 604 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 604 according to various correlations; correlations may indicate causative and/or predictive links between categories of data elements, which may be modeled as relationships such as mathematical relationships by machine-learning processes as described in further detail below. Training data 604 may be formatted and/or organized by categories of data elements, for instance by associating data elements with one or more descriptors corresponding to categories of data elements. As a non-limiting example, training data 604 may include data entered in standardized forms by persons or processes, such that entry of a given data element in a given field in a form may be mapped to one or more descriptors of categories. Elements in training data 604 may be linked to descriptors of categories by tags, tokens, or other data elements; for instance, and without limitation, training data 604 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 6, training data 604 may include one or more elements that are not categorized; that is, training data 604 may not be formatted or contain descriptors for some elements of data. Machine-learning algorithms and/or other processes may sort training data 604 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 604 to be made applicable for two or more distinct machine-learning algorithms as described in further detail below. Training data 604 used by machine-learning module 600 may correlate any input data as described in this disclosure to any output data as described in this disclosure. As a non-limiting illustrative example inputs may include any inputs as described above, such as but not limited to conduct indicators and outputs may include outputs as described above such as but not limited to functional signatures.

Further referring to FIG. 6, training data may be filtered, sorted, and/or selected using one or more supervised and/or unsupervised machine-learning processes and/or models as described in further detail below; such models may include without limitation a training data classifier 616. Training data classifier 616 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 600 may generate a classifier using a classification algorithm, defined as a processes whereby a computing device and/or any module and/or component operating thereon derives a classifier from training data 604. Classification may be performed using, without limitation, linear classifiers such as without limitation logistic regression and/or naïve Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 616 may classify elements of training data to subgroupings of conduct indicators wherein subgroupings may include foods, health activities, religious activities and the like.

Still referring to FIG. 6, computing device 604 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)+P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 604 may then calculate a likelihood table by calculating probabilities of different data entries and classification labels. Computing device 604 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 6, computing device 604 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 6, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {{a}_{i}}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 6, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 6, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 6, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 6, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 6, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 6, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 6, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 6, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{max} : {X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Further referring to FIG. 6, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 6, machine-learning module 600 may be configured to perform a lazy-learning process 620 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 604. Heuristic may include selecting some number of highest-ranking associations and/or training data 604 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 6, machine-learning processes as described in this disclosure may be used to generate machine-learning models 624. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 624 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 624 may be generated by creating an artificial neural network, such as a convolutional neural network comprising an input layer of nodes, one or more intermediate layers, and an output layer of nodes. Connections between nodes may be created via the process of “training” the network, in which elements from a training data 604 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 6, machine-learning algorithms may include at least a supervised machine-learning process 628. At least a supervised machine-learning process 628, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs such as conduct indicator as described above as inputs, outputs such as functional signature as described above as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 604. Persons skilled in the art, upon reviewing the entirety of this disclosure, will be aware of various possible variations of at least a supervised machine-learning process 628 that may be used to determine relation between inputs and outputs. Supervised machine-learning processes may include classification algorithms as defined above.

With further reference to FIG. 6, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 6, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 6, machine learning processes may include at least an unsupervised machine-learning processes 632. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 632 may not require a response variable; unsupervised processes 632 may be used to find interesting patterns and/or inferences between variables, to determine a degree of correlation between two or more variables, or the like.

Still referring to FIG. 6, machine-learning module 600 may be designed and configured to create a machine-learning model 624 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 6, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 6, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 6, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 6, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 6, one or more processes or algorithms described above may be performed by at least a dedicated hardware unit 636. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 636 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 636 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 636 to perform one or more operations described herein, such as evaluation of model and/or algorithm outputs, one-time or iterative updates to parameters, coefficients, weights, and/or biases, and/or any other operations such as vector and/or matrix operations as described in this disclosure.

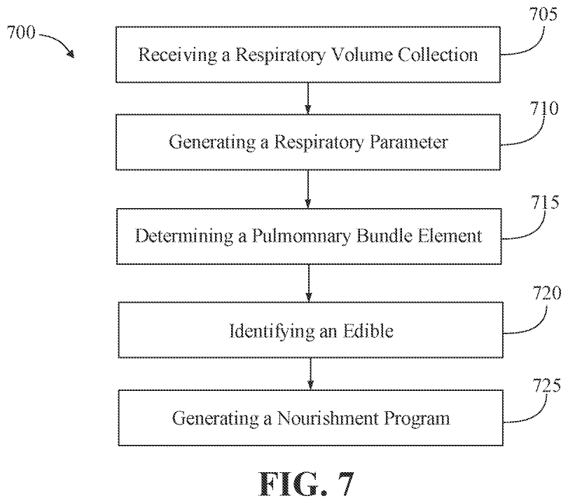

Now referring to FIG. 7, an exemplary embodiment of a method 700 for generating a pulmonary dysfunction nourishment program is illustrated. At step 705, a computing device 104 receives a respiratory collection 108. Computing device 104 includes any of the computing device 104 as described above, in reference to FIGS. 1-6. Respiratory volume collection 108 includes any of the respiratory volume collection 108 as described above, in reference to FIGS. 1-6. For instance, and without limitation, respiratory volume collection 108 may include one or more breath and/or blood samples provide by a user.

Still referring to FIG. 7, at step 710, computing device 104 generates at least a respiratory parameter 112 of a plurality of respiratory parameters as a function of respiratory volume collection 108. Respiratory parameter 112 includes any of the respiratory parameter 112 as described above, in reference to FIGS. 1-6. Computing device 104 generates respiratory parameter 112 using a respiratory algorithm 116. Respiratory algorithm 116 includes any of the respiratory algorithm 116 as described above, in reference to FIGS. 1-6.

Still referring to FIG. 7, at step 715, computing device 104 determines a pulmonary bundle element 120 as a function of respiratory parameter 112. Pulmonary bundle element 120 includes any of the pulmonary bundle element 120 as described above, in reference to FIGS. 1-6.

Still referring to FIG. 7, at step 720, computing device 104 identifies at least an edible 124 as a function of pulmonary bundle element 120. Edible 124 includes any of the edible 124 as described above, in reference to FIGS. 1-6. Edible 124 is identified by obtaining a nourishment composition 128 from an edible directory 132. Nourishment composition 128 includes any of the nourishment composition 128 as described above in reference to FIGS. 1-6. Edible directory 132 includes any of the edible directory 132 as described above, in reference to FIGS. 1-6. Edible 124 is identified by determining a nourishment deficiency 136 as a function of pulmonary bundle element 120. Nourishment deficiency 136 includes any of the nourishment deficiency 136 as described above, in reference to FIGS. 1-6. Edible 124 is identified using nourishment composition 128, nourishment deficiency 136, and an edible machine-learning model 140. Edible machine-learning model 140 includes any of the edible machine-learning model 140 as described above, in reference to FIGS. 1-6.

Still referring to FIG. 7, at step 725, computing device 104, outputs a nourishment program 144 of a plurality of nourishment programs as a function of edible 124. Nourishment program 144 includes any of the nourishment program 144 as described above, in reference to FIGS. 1-6.

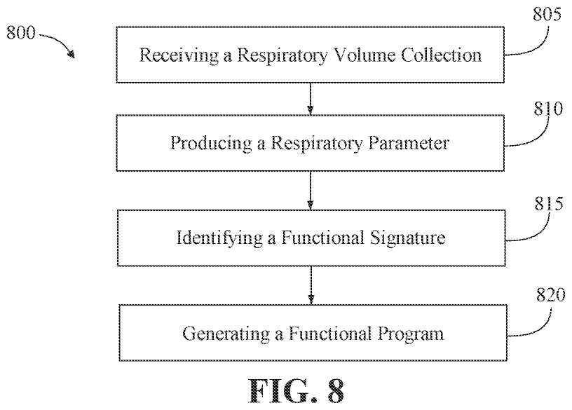

Now referring to FIG. 8, an exemplary embodiment of a method 800 for generating a pulmonary dysfunction functional program is illustrated. At step 805, a computing device 104 receives a respiratory collection 108. Computing device 104 includes any of the computing device 104 as described above, in reference to FIGS. 1-7. Respiratory volume collection 108 includes any of the respiratory volume collection 108 as described above, in reference to FIGS. 1-7. For instance, and without limitation, respiratory volume collection 108 may include one or more breath and/or blood samples provide by a user.

Still referring to FIG. 8, at step 810, computing device 104 produces at least a respiratory parameter 112 of a plurality of respiratory parameters as a function of respiratory volume collection 108. Respiratory parameter 112 includes any of the respiratory parameter 112 as described above, in reference to FIGS. 1-7. Computing device 104 generates respiratory parameter 112 using a respiratory algorithm 116. Respiratory algorithm 116 includes any of the respiratory algorithm 116 as described above, in reference to FIGS. 1-7.

Still referring to FIG. 8, at step 815, computing device 104 identifies functional signature 204. Functional signature 204 includes any of the functional signature 204 as described above, in reference to FIGS. 1-7. Computing device 104 identifies functional signature 204 as a function of receiving a conduct indicator 208. Conduct indicator 208 includes any of the conduct indicator 204 as described above, in reference to FIGS. 1-7. Computing device 104 identifies functional signature 204 as a function of conduct indicator 208 and pulmonary bundle element 120 using a functional machine-learning model 212. Functional machine-learning model 212 includes any of the functional machine-learning model 212 as described above, in reference to FIGS. 1-7.

Still referring to FIG. 8, at step, 820, computing device 104 generates a functional program 216 as a function of functional signature 204. Functional program 216 includes any of the functional program 216 as described above, in reference to FIGS. 1-7.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

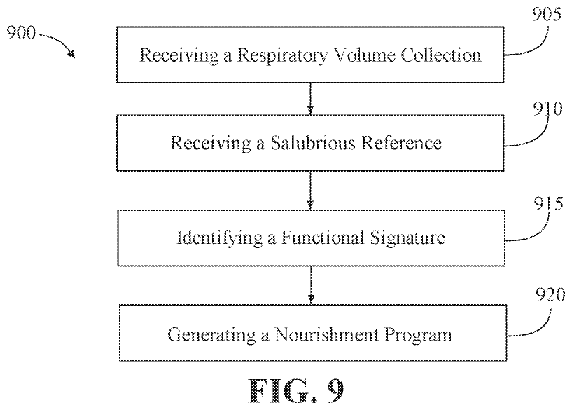

Referring now to FIG. 9, an exemplary method 900 of generating a pulmonary dysfunction nourishment program is described. At step 905, method 900 includes receiving, by a computing device, at least a respiratory volume collection relating to a user, wherein the at least respiratory volume collection includes at least a biomarker, wherein the at least biomarker comprises a bio remnant and a volatile organic compound (VOC). This may be implemented with reference to FIGS. 1-9 and without limitation.

With continued reference to FIG. 9, at step 910, method 900 includes receiving, by the computing device, a salubrious reference relating to the user, wherein the salubrious reference includes a blood pressure reference. This may be implemented with reference to FIGS. 1-9 and without limitation.

With continued reference to FIG. 9, method 915 includes identifying, by the computing device, a functional signature as a function of the salubrious reference, wherein the identifying further includes receiving a conduct indicator wherein the conduct indicator includes an indicator index, generating one or more conduct parameters as function of the salubrious reference, comparing the conduct indicator to the one or more conduct parameters identifying the functional signature as a function of the comparison. This may be implemented with reference to FIGS. 1-9 and without limitation.

With continued reference to FIG. 9, at step 920 method 900 includes generating by the computing device a nourishment program as a function of the functional signature. This may be implemented with reference to FIGS. 1-9 and without limitation.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

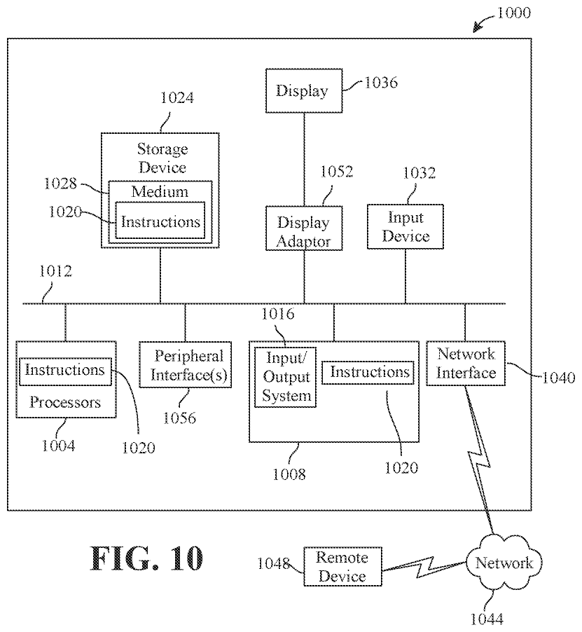

FIG. 10 shows a diagrammatic representation of one embodiment of a computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and a memory 1008 that communicate with each other, and with other components, via a bus 1012. Bus 1012 may include any of several types of bus structures including, but not limited to, a memory bus, a memory controller, a peripheral bus, a local bus, and any combinations thereof, using any of a variety of bus architectures.

Processor 1004 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1004 may be organized according to Von Neumann and/or Harvard architecture as a non-limiting example. Processor 1004 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1008 may include various components (e.g., machine-readable media) including, but not limited to, a random-access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 13104 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, etc.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computer system 1000 via network interface device 1040.

Computer system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve systems and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for generating a pulmonary dysfunction nourishment program, the system comprising:
a computing device, the computing device configured to:
receive at least a respiratory volume collection relating to a user, wherein the at least respiratory volume collection comprises at least a biomarker, wherein the at least biomarker comprises a bioremnant and a volatile organic compound (VOC);
receive a salubrious reference relating to the user;
identify a functional signature as a function of the salubrious reference, wherein the identifying further comprises:
receiving a conduct indicator, wherein the conduct indicator comprises an indicator index;
generating one or more conduct parameters as a function of the salubrious reference;
comparing the conduct indicator to the one or more conduct parameters; and
identifying the functional signature as a function the comparison; and
generate a nourishment program as a function of the functional signature.

2. The system of claim 1, wherein the indicator index comprises a measurable value associated with the conduct indicator.

3. The system of claim 1, wherein generating the one or more conduct parameters comprises:
selecting at least one conductor parameter from a plurality of conduct parameters as a function of user physiological data; and
comparing the conductor indicator to the at least one conduct parameter.

4. The system of claim 1, wherein receiving the conduct indicator comprises automatically receiving the conduct indicator from a wearable device belonging to the user.

5. The system of claim 1, wherein the computing device is further configured to periodically transmit the nourishment program to a remote device associated with the user.

6. The system of claim 1, wherein the computing device is further configured to select at least one nourishment coach as a function of the nourishment program.

7. The system of claim 1, wherein receiving at least the respiratory volume collection relating to the user comprises iteratively receiving at least the respiratory volume from one or more medical entities.

8. The system of claim 1, wherein the computing device is further configured to generate a graphical representation of the functional signature and a plurality of functional signatures received from previous iterations of the processing.

9. The system of claim 6, wherein the at least one nourishment coach comprises a large language model.

10. The system of claim 1, wherein generating the nourishment program comprises generating at least one predicted functional signature as a function of an adherence to the nourishment program.

11. A method for generating a pulmonary dysfunction nourishment program, the method comprising:
receiving, by a computing device, at least a respiratory volume collection relating to a user, wherein the at least respiratory volume collection comprises at least a biomarker, wherein the at least biomarker comprises a bioremnant and a volatile organic compound (VOC);
receiving, by the computing device, a salubrious reference relating to the user;
identifying, by the computing device, a functional signature as a function of the salubrious reference, wherein the identifying further comprises:
receiving a conduct indicator, wherein the conduct indicator comprises an indicator index;
generating one or more conduct parameters as function of the salubrious reference;
comparing the conduct indicator to the one or more conduct parameters;
identifying the functional signature as a function of the comparison; and
generating, by the computing device, a nourishment program as a function of the functional signature.

12. The method of claim 11, wherein the indicator index comprises a measurable value associated with the conduct indicator.

13. The method of claim 11, wherein generating the one or more conduct parameters comprises:
selecting at least one conductor parameter from a plurality of conduct parameters as a function of user physiological data; and
comparing the conductor indicator to the at least one conduct parameter.

14. The method of claim 11, wherein receiving the conduct indicator comprises automatically receiving the conduct indicator from a wearable device belonging to the user.

15. The method of claim 11, wherein the method further comprises transmitting, by the computing device, the nourishment program to a remote device associated with the user.

16. The method of claim 11, wherein the method further comprises selecting, by the computing device, at least one nourishment coach as a function of the nourishment program.

17. The method of claim 11, wherein receiving, by the computing device, at least the respiratory volume collection relating to the user comprises iteratively receiving at least the respiratory volume from one or more medical entities.

18. The method of claim 11, wherein the method further comprises, generating by the computing device, a graphical representation of the functional signature and a plurality of functional signatures received from previous iterations of the processing.

19. The method of claim 16, wherein the at least one nourishment coach comprises a large language model.

20. The method of claim 11, wherein generating, by the computing device, the nourishment program comprises generating at least one predicted functional signature as a function of an adherence to the nourishment program.
